(19) United States
(12) Patent Application Publication
(10) Pub. No.: US 2009/0253771 A1
(43) Pub. Date: Oct. 8, 2009

(54) INHIBITION OF GLIOTOXIN

(75) Inventors: Markus M. Simon, Freiburg (DE); Julian Pardo, Zaragoza (ES)

Correspondence Address: ROTHWELL, FIGG, ERNST & MANBECK, P.C., 1425 K STREET, N.W., SUITE 800, WASHINGTON, DC, 20005, US

(73) Assignee: Max-Planck-Gesellschaft zur Foerderung der Wissenschaften e.V, Muenchen (DE)

(21) Appl. No.: 12/298,798

(22) PCT Filed: May 7, 2007
(86) PCT No.: PCT/EP2007/004023

§ 371 Date: Oct. 28, 2008

(30) Foreign Application Priority Data
May 8, 2006 (EP) 06009460.4

(51) Int. Cl. A61K 31/7105; G01N 33/68; C12Q 1/02; C12N 5/06; C12Q 1/18; A61P 31/10

(52) U.S. Cl. 514/44 A; 436/86; 435/29; 435/325; 435/32

(57) ABSTRACT

The present invention relates to the inhibition of the interaction between Gliotoxin (GT) and its intracellular target for the prevention and/or treatment of fungal infections. Further, novel methods and systems for identifying antifungal agents are disclosed.

The present invention relates to the inhibition of the interaction between Gliotoxin (GT) and its intracellular target for the prevention and/or treatment of fungal infections. Further, novel methods and systems for identifying antifungal agents are disclosed.

A. fumigatus is responsible for >90% of invasive aspergilloses (IA) with mostly fatal outcomes in immuno-compromised patients suffering from AIDS, tuberculosis, cancer or bone marrow/organ transplants [Latge, 1999], yet the pathobiology of this opportunistic pathogen and its critical virulence factors are only poorly understood. Gliotoxin (GT), when identified as an immunosuppressive agent, has been proposed to constitute a virulence factor in IA [Mullbacher, 1984; Eichner, 1984; Lewis, 2005]. Evidence for increased virulence of A. fumigatus as a result of GT has since been obtained in vivo [Sutton, 1996; Latge, 2001; Stanzani, 2005; Bok, 2005; and own unpublished results]. GT belongs to the epipolythiodioxipiperazine (ETP) class of secondary fungal metabolites [Taylor, 1971] and was shown to induce mammalian cell apoptosis [Waring, 1988; Sutton, 1994] accompanied by the production of reactive oxygen species (ROS) and mitochondrial membrane disruption [Eichner, 1988; Suen, 2001; Zhou, 2000]. To identify the intracellular target of GT and to further validate the mitochondrial pathway as the crucial mediator for its cytotoxic action, we used a panel of mouse embryo fibroblasts (MEFs) and IL-3-(factor) dependent mouse monocytes (FDMs) deficient in the putative pore-forming Bcl-2 family members Bax or Bak or their activator Bid [Wei, 2001]. As GT may induce apoptosis and necrosis in mammalian cells, depending on the concentration of GT and the target cell used [Kweon, 2003; Orr, 2004], we first established optimal conditions that allowed us to readily monitor pro-apoptotic processes in MEFs.

In the present application it is shown that Gliotoxin (GT) induces apoptotic cell death by selectively activating the pro-apoptotic Bcl-2 family member Bak, but not Bax to elicit the generation of reactive oxygen species, the mitochondrial release of apoptogenic factors and caspase-3 activation. Activation of Bak by GT seems to be direct as GT can trigger in vitro a dose-dependent release of cytochrome c from purified mitochondria isolated from wild-type and Bax—but not from Bak-deficient cells. Resistance to A. fumigatus of mice lacking Bak compared to wild-type mice demonstrates the in vivo relevance of this GT-induced apoptotic pathway involving Bak. These results show a correlation between GT production and virulence of A. fumigatus. The elucidation of its molecular basis, as presented here, opens new strategies for the development of potential therapeutic regimens to combat A. fumigatus and related fungal infections in compromised patients.

A first aspect of the present invention is the use of an inhibitor of the Gliotoxin (GT)-mediated activation of the pro-apoptotic Bcl-2 family member Bak for the manufacture of a medicament for the prevention and/or treatment of fungal infections.

A further aspect of the present invention relates to a method of identifying antifungal agents comprising determining whether a compound is capable of inhibiting the interaction between GT and Bak.

Still a further subject-matter of the present application is a test system for identifying antifungal agents comprising a Fas-negative cell.

According to the first aspect of the invention, an inhibitor of GT-mediated activation or Bak is provided. In a preferred embodiment, the inhibitor is capable of suppressing GT-mediated pro-apoptotic and/or apoptotic processes. More preferably, the inhibitor selectively suppresses GT-faciliated activation of pro-apoptotic Bak and is substantially inactive against other pro-apoptotic proteins, particularly pro-apoptotic proteins of the Bcl-2 family such as Bax. Even more preferably, GT-mediated activation of Bak proceeds via modulation of the voltage-dependent anion channel 2 (VDAC2). Inhibition of Bak activation may be determined with conformation-specific antibodies against the N-terminus of Bak, which is exposed and thus immuno-reactive after activation. For example, the inhibitor is capable of an at least partial inhibition of the GT-mediated generation of reactive oxygen species, the mitochondrial release of apoptogenic factors and/or caspase-3 activation mediated by Bak. Suitable methods for determining inhibition of GT-mediated Bak activation and Bak-faciliated pro-apoptotic and/or apoptotic processes are as described in the Examples.

In an especially preferred embodiment of the invention, the inhibitor binds to GT, VDAC2 and/or to Bak. For example, the inhibitor may be a polypeptide molecule which specifically binds to GT, VDAC2 and/or to Bak, e.g. an antibody, an anticalin, a scaffold molecule, an aptamer, a spiegelmer or a chemical compound directed against GT or against Bak. Preferred inhibitors are antibodies such as monoclonal antibodies, chimeric antibodies, humanized antibodies or antigen-binding fragments thereof as well as recombinant antibodies, e.g. single-chain antibodies or single-chain antibody fragments. Suitable antibodies may by generated by standard procedures known in the art. Further preferred inhibitors are anticalins which represent engineered receptor proteins with antibody-like ligand-binding functions derived from natural lipocalins as a scaffold [Beste; 1999; Skerra, 2001; Schlehuber, 2001]. A further preferred inhibitor is Vaccina virus protein F1L which binds to Bak and is capable of inhibiting Bak-induced apoptotic processes [Wasilenko, 2005].

In a further preferred embodiment, the inhibitor is a nucleic acid molecule which inhibits Bak expression, e.g. an antisense molecule, a ribozyme or a nucleic acid molecule capable of RNA interference, e.g. a siRNA molecule, or a DNA molecule encoding such a nucleic acid inhibitor molecule.

The inhibitor may be used for the prevention and/or treatment of fungal infections, e.g. infections with Aspergillus species, e.g. A. fumigatus and/or Candida species, e.g. C. albicans or Thermoascus crustacus. The inhibitor may be used in human or veterinary medicine. Particularly preferred is the use in immuno-compromised or immuno-suppressed patients, e.g. patients infected with HIV, tuberculosis, cancer, patients undergoing organ transplantation, or those with autoimmune sequelae.

Further, the invention relates to a screening method for identifying antifungal agents. In this method it is determined whether a test compound is capable of inhibiting the interaction between GT and Bak. The test compound may be derived from a chemical library of compounds. Preferably, the method is a High Throughput Screening Method wherein a plurality of test compounds is screened in parallel. A compound which exhibits a significant inhibition of the GT-Bak interaction is a suitable candidate antifungal agent.

The method may be any molecular screening method or cellular screening method which allows determining the effect of a test compound on the GT-Bak interaction with a suitable detection technology. The range of assay technologies supported for formatting molecular screens may include AlphaScreen, time resolved fluorescence (DELPHIA, and LANCE), fluorescence polarisation, steady-state fluorescence, photometry, chemiluminescence, ELISA, scintillation proximity, and filtration-based separations. For cell-based screens, supported assays may include reporter genes (luciferase, fluorescent proteins, alkaline phosphatase, beta-galactosidase), BRET (protein-protein interactions), or assays measuring biochemical responses such as cell-surface antigen expression, cytokine expression, cell proliferation and cytotoxicity.

In a preferred embodiment, the method is a cellular screening method wherein it is determined whether a test compound is capable of suppressing pro-apoptotic and/or apoptotic processes mediated by an interaction between GT and Bak in a suitable cell or cell line. Especially preferred is the use of a Fas-negative cell line, i.e. a cell line, preferably a mammalian and/or a tumor cell line, which is resistant to Fas-mediated apoptosis, e.g. the methylcholanthrene-induced cell-line MC.Fas^{-/-} which was generated in the inventors' own laboratory by A. Mullbacher and deposited at the DSMZ (date of deposition: 4 May 2006) according to the Budapest treaty.

According to the present invention it was found that GT is able to introduce pro-apoptotic processes in Fas-negative cells in a dose-dependent manner, resulting in loss of cell adhesion and in cell death. This pro-apoptotic activity may be inhibited by GT-inhibitors, e.g. anti-GT-antibodies. The degree of cell adhesion and/or cell death may be determined by standard methods. For example, the cells may be labelled with a suitable dye, e.g. neutral red, allowed to adhere and incubated subsequently with GT in the presence of a test compound. After an appropriate incubation time, the non-adherent cells may be removed and the remaining adherent cells and/or the removed cells may be determined. For example, the dye neutral red may be released from cells by acidic acid/ethanol. Non-adherent cells equating with cell death may be quantitatively determined, e.g. with an optical detection method, preferably by measuring the absorbance at 540 nm according to standard methods, e.g. in a microplate reader.

Still further, the invention provides a test system for identifying antifungal cells comprising a Fas-negative cell line as described above, optionally in combination with labelling reagents such as dyes. In a preferred embodiment, the Fas-negative cell line is a mammalian and/or a tumor cell line.

Finally, the invention relates to the cell line MC.Fas^{-/-} (date of deposition at DSMZ: 4 May 2006).

The invention shall be explained further by the following figures and examples.

### FIGURE LEGENDS

FIG. 1: Gliotoxin induces apoptosis in mouse embryonic fibroblasts (MEFs). Wild-type (wt) MEFs were incubated with or without 1 μM GT for 4 h and stained with 10 μg/ml of Hoechst 33342 (A) or annexin V-FITC plus propidium iodide (PI) (B). Cells were analysed by fluorescence microscopy (Axioskop, Zeiss). Magnification 400× (A) or 1000× (B).

FIG. 2: Gliotoxin-induced apoptosis-inducing factor (AIF) translocation to the nucleus is dependent on Bak and the generation of ROS. (A) wt, Bak^{-/-}, Bax^{-/-} and Bak×Bak^{-/-} MEF cells were incubated with or without 1 μM GT for 4 h and analysed by confocal microscopy for nuclear translocation of AIF using a specific anti-AIF rabbit antibody. Numbers depicted are the percentages of cells with nuclear AIF. (B) Alternatively, wt MEFs were incubated with or without 1 μM of GT for 4 h in the presence or absence of 100 μM Z-VAD.fmk or 15 mM NAC and analyzed for nuclear translocation of AIF as described above (magnification ×400).

FIG. 3: Proposed mechanism for gliotoxin-induced cell death and Aspergillosis treatment. After production by A. fumigatus, gliotoxin (GT) would enter cells by a redox-dependent mechanism and directly induce a conformational change in Bak leading to mitochondrial depolarization and ROS production. ROS production then triggers the mitochondrial release of apoptogenic proteins such as cytochrome c (cyt c) and AIF. In this way, both caspase-dependent and -independent processes would be launched to induce cell death. By blocking either GT and/or Bak conformational change and/or ROS production, GT-induced cell death could be prevented and the damage exerted by A. fumigatus attenuated.

FIG. 4: Gliotoxin-induced apoptosis is Bak-dependent. (A,B) Wild-type (wt), Bak^{-/-}, Bax^{-/-}, Bak×Bax^{-/31} and Bid^{-/-} MEFs were incubated with or without 1 μM GT for 4 h and analysed by FACS for PS exposure (annexin V-FITC) and PI uptake (PI) (A) or Δψₘ loss (DiOC₆(3)) and ROS generation (2-HE) (B). The same cells were incubated with increasing amounts of GT for 4 h to determine the percentage of cell death (trypan blue exclusion) and cell detachment (C) by microscopic inspection. Values are the means±sd; ns: no significant difference (analysed by unpaired two-tailed t-test comparing wt with Bak^{-/-}×Bax^{-/- or Bak}^{-/-}; no of exp.=4). Data shown in A-C are representatives of at least four independent experiments with similar outcome.

FIG. 5: (A,B) Gliotoxin induces conformational change of Bak, but not of Bax in MEFs. wt and Bak^{-/-}×Bax^{-/-} MEFs were incubated for 4 h without (black) or with (red) either GT (1 μM; A) or staurosporine (1 and 5 μM; B) and analysed by FACS for conformational changes of Bak and Bax using mAb specific for the functionally active N-terminal region in each protein. Numbers depicted are the percentages of cells positive for active Bak or Bax (indicated by the horizontal bars).

(C-E) Gliotoxin-induced conformational change of Bak is independent of caspase activation, ROS generation and the presence of Bid. (C) The ROS scavengers NAC and MnTBAP were tested for their efficiency in inhibiting ROS production by FACS analysis (2-HE) of wt MEFs treated with 1 μM for 4 h. To test for the caspase-inhibiting potency of Z-VAD.fmk, MBL. Fas cells were treated with 1 μg/ml of the anti-Fas antibody Jo-2 in the absence and presence of 100 μM Z-VAD.fmk and cell death monitored by trypan blue exclusion. (D) wt MEFs were incubated with (red) or without (black) 1 μM GT for 4 h in the presence or absence of 100 μM Z-VAD.fmk (green) or 15 mM of either NAC or MnTBAP. The cells were analysed by FACS for conformational changes of Bak as under (A). (E) GT-induced conformational change of Bak is independent of Bid. wt and Bid^{-/-} MEFs were treated with GT and FACS analyzed for conformational changes of Bak as under (A). Numbers depicted are the percentages of cells positive for active Bak (indicated by the horizontal bars). Data shown are representative of at least three independent experiments with similar outcome.

FIG. 6: Gliotoxin-induced mitochondrial membrane perturbation and apoptosis is dependent on ROS generation while only the latter partially depends on caspases. (A) wt MEFs were incubated with or without 1 μM GT for 4 h in the presence or absence of 100 μM of the pan-caspase inhibitor ZVAD-fmk (green) or 15 mM of the ROS scavenger NAC and analysed by FACS for PS exposure (annexin V-FITC)/PI uptake and Δψₘ loss (DiOC₆(3))/ROS generation (2-HE). (B) wt MEFs were incubated with increasing amounts of GT for 4 h in the presence or absence of ZVAD-fmk or NAC and the percentage of cell death (trypan blue exclusion) and cell detachment was determined by microscopic inspection. Values are the means±sd; ns: no significant difference. **: significantly different (analysed by unpaired two-tailed t-test comparing medium and ZVAD-fmk or NAG; no of exp.=4, p=0,0745 ZVAD-fmk, p=0,0063 NAC); (C) wt, Bak^{-/-}, Bax^{-/-} and Bak^{-/-}×Bax^{-/-} MEFs were incubated with (red) or without (black) 1 μM GT for 4 h and analysed by FACS for the activation of caspase 3, using a monoclonal anti-caspase-3 antibody against the active form of the enzyme followed by a FITC-labeled secondary antibody. Data shown are representatives of at least four independent experiments with similar outcome.

FIG. 7: Gliotoxin-induced cytochrome c release from mitochondria is dependent on Bak and the ROS generation. (A) wt, Bak^{-/-}, Bax^{-/-} and Bak^{-/-}×Bax^{-/-} MEFs were incubated with (red) or without (black) 1 μM GT for 4 h and analysed by FACS for mitochondrial release of cytochrome c using a monoclonal anti-cytochrome c antibody followed by a FITC-labeled secondary antibody. Numbers depicted are the percentages of cells negative for cytochrome c (indicated by horizontal bars). (B) Mitochondria isolated from wt, Bax^{-/-}, Bak^{-/-} and Bak^{-/-}×Bax^{-/-} FDMs (left panels) or MEFs (right panels) were treated with 10, 20 or 50 μM of GT or 40 nM of tBid for 4 h in vitro. After centrifugation cytochrome c was measured in mitochondria and the supernatant by anti-cytochrome c Western blotting. (C) wt MEF cells were incubated with (red) or without (black) 1 μM GT for 4 h in the presence or absence of 100 μM ZVAD-fmk (green), 15 mM NAC (blue) and analysed by FACS for mitochondrial release of cytochrome c as under (A). Data shown are representatives of at least three independent experiments with similar outcome.

FIG. 8: (A), Bak^{-/-} mice are resistant to A. fumigatus infection. Female wt C57BL6 or Bak^{-/-} mice (6 mice per group) were immuno-suppressed by subcutaneous injection of 2 mg of hydrocortisone in PBS/0.1% Tween-20 on days −4, −2, 0, 2 and 4 of infection. On day 0, recipients were inoculated intranasally with 5'10⁶ A. fumigatus B5233 in 20 μl of PBS or 20 μl PBS alone (control) and morbidity and mortality was monitored during the time. Control mice survive without any symptoms of infection. (B), A. fumigatus B5233 is able to produce gliotoxin. 1×10⁷ A. fumigatus spores were inoculated in 100 ml of RPMI and grown for 48 h at 37° C., 0,5% CO₂. After that, gliotoxin was analysed as described in methods. Gliotoxin eluated after 13 min (arrow) as detected by comparison with a gliotoxin standard.

### EXAMPLE 1

### Materials and Methods

1. Cell Culture and Reagents

SV40 transformed mouse embryonic fibroblasts (MEFs) [Wei, 2001] and MBL-2.Fas cells [van den Broek, 1996] were cultured in MEM supplemented with 10% FCS and 2-mercaptoethanol (10⁻⁵ M) at 37° C., 7% CO₂. The IL-3 (factor) dependent cell lines (FDMs) were generated by co-culturing E14.4 fetal liver single cell suspensions with fibroblasts expressing a HoxB8 retrovirus in the presence of high IL-3 concentrations, as previously described [Ekert, 2004]. Bak^{-/-} mice [Wei, 2000] were back-crossed for further 9 generations to ensure a “pure” C57BL/6 genetic background and then intercrossed with Bax^{-/-} C57BL/6 mice [Lindsten, 2000] to obtain Bax^{-/-}×Bak^{-/-} mice as described [Willis, 2005]. The cell lines were cultured in DMEM with 10% FCS supplemented with IL-3.

Gliotoxin (GT) was purified from Penicillium terlikowskii as described [Waring, 1988]. For apoptosis induction, 4×10⁵ MEFs were incubated with different concentrations of GT or staurosporine (Sigma) for 4 h and apoptosis assays were performed as described below. In some cases the general caspase inhibitor Ac-ZVAD-fmk (Bachem) or the ROS scavengers N-acetylcysteine (NAC) (Sigma) or MnTBAP (Calbiochem) were added as described [Pardo, 2004]. To test the inhibitory potency of Ac-ZVAD-.fmk, MBL-2. Fas cells were incubated with anti-Fas mAb Jo-2 (1 μg/ml) for 24 h in the presence or absence of 100 μM of the caspase inhibitor and cell death was analysed by trypan blue exclusion. Nuclei were stained with 10 μg/ml of Hoechst 33342 (Molecular Probes).

2. Plasma and Mitochondrial Membrane Perturbations

Phosphatidylserine (PS) exposure and propidium iodide (PI) uptake was analysed by FACS or fluorescence microscopy as described [Pardo, 2004] using the annexin V-FITC kit from BD Pharmingen. The mitochondrial membrane potential was measured with the fluorescent probe 3,3′-dihexyloxacarbocyanine iodide (DiOC₆(3), Molecular Probes) and ROS generation with 2-hydroxiethidine (2-HE, Molecular Probes) as described [Pardo, 2004].

3. Caspase-3 activation

Cells were fixed with 4% paraformaldehyde (PFA) and incubated with a monoclonal antibody against the active form of caspase-3 (clone C92605, BD Pharmingen). Following incubation with a FITC-labeled secondary antibody, the cells were analyzed by FACS as described [Pardo, 2004].

4. Cytochrome C Release and Nuclear Translocation of AIF on Cells

Cytochrome c release was quantified by FACS analysis as recently described [Waterhouse, 2003]. Briefly, 1×10⁶ of MEFs were mildly permeabilized with 25 μg/ml digitonin plus 100 mM KCl on ice for 5 min. This lead to the cellular loss of cytosolic cytochrome c. Cells were washed once with cold PBS, fixed in 4% PFA, permeabilized with 0.05% saponin and 3% BSA and then incubated with the anti-cytochrome c mAb 6H2.B4 (BD Pharmingen) or mouse IgG isotype control (Jackson) followed by anti-mouse-FITC secondary antibody (Jackson). The cells were resuspended in 100 μl PFA in PBS and analysed by FACS with a FACScan (BD) and CellQuest® software. For the analysis of the nuclear translocation of AIF, cells were fixed, mounted on poly-L-lysine coverslides and stained with a rabbit polyclonal antiAIF antibody (Sigma) as described [Pardo, 2001]. Afterwards the cells were analysed by confocal microscopy using a Leica SP2 confocal microscope and Imaris® software.

5. Cytochrome C Release from Isolated Mitochondria

8×10⁷ MEFs or FDMs were centrifuged and washed once in PBS. The cell pellets were resuspended in 500 μl MSH-Buffer (210 mM mannitol; 70 mM sucrose; 20 mM HEPES; 1 mM EDTA; pH 7,5; 100 μM PMSF; 400 ng/ml pepstatin; 10 μg/ml leupeptin; 10 μg/ml aprotinin and 5 μg/ml cytochalasin B). The resuspended cell pellet was incubated on ice for 15 min before the cells were broken by passaging 25 times through a 23-gauge needle. The lysate was centrifuged at 500×g for 5 min to remove cell debris and nuclei. A crude mitochondrial pellet was then obtained by centrifugation at 10,000×g for 15 min and resuspended in MSH-Buffer. The isolated mitochondria were incubated with different concentrations of GT (10 μM, 20 μM and 50 μM), or 40 nM of recombinant tBid as a positive control at 37° C. for 4 hr. Following incubation, the mitochondria were pelleted, and both, pellet and supernatant were tested for cytochrome c release by SDS-PAGE.

6. Conformational Change of Bax and Bak

MEFs were fixed in 4% PFA, permeabilized with 0.1% saponin in PBS/5% fetal calf serum (FCS) and incubated with 2 μg/ml rabbit polyclonal anti-Bak (NT, Upstate Biotechnology) 5 μg/ml rabbit polyclonal anti-Bax (NT, Upstate Biotechnology) or 5 μg/ml rabbit purified IgG (control). After two washes with 0.1% saponin in PBS, the cells were incubated with anti rabbit-FITC antibody in 0.1% saponin/PBS/5% FCS, washed twice in 0.1% saponin/PBS, resuspended in 1% PFA/PBS and analysed by FACS with a FACScan (BD) and CellQuest® software.

7. In vivo Invasive Aspergillosis Model and Gliotoxin Analysis on Culture Supernatants

Mice (C57BL/6, Bak^{-/-}, Jackson, C57BL/6.129, 6 times backcrossed in C57BL/6 or 129, female) were immuno-suppressed by subcutaneous injection of 3 mg (112 mg/kg) of hydrocortisone (Sigma) diluted in 200 μl of PBS/0.1% Tween 20 on days −4, −2, 0, 2 and 4, as described [Tang, 1993]. On day 0 mice (6 per group) were infected intranasally with 5×10⁶ Aspergillus fumigatus B5233 conidia in 20 μl of PBS or with PBS alone. Disease development was analysed by morbidity/mortality of the mice after infection. There was no difference in the sensitivity of C57BL/6 or 129 mice to Aspergillus infection. All infected animals died during the first week after infection. Gliotoxin presence on Aspergillus Fumigatus B5233 culture supernatants was analysed after 48 h by HPLC as described [Belkacemi, 1999].

### EXAMPLE 2

### Manufacture of Mammalian Tumor Cell Line MC.Fas^{-/-} (Date of Deposition at DSMZ: 4 May 2006)

100 μl of olive oil were added to 0.5 mg of 20-methylcholanthrene (Sigma M-6501) placed in a small vial. After stirring by means of a magnetic stirrer and gently heating for about 2 hours the solution thus obtained was injected intramuscularly (i.m.) into two sites of a Fas knockout mouse strain [Adachi, 1995]. In general, about 6-12 weeks after injection the first tumours appeared. In the case of the mice not developing visible tumours it might, however, be necessary to repeat the treatment described above.

After harvesting the tumours were introduced into the neomycin-containing cell line H16 according to the trypsin method (˜98% viability). For this purpose, the tumours were initially cut into small pieces, and the pieces were put into a small bottle with a stirrer bar in it. After adding 9 ml of H16, 1 ml of 2.5% trypsin and 100 mg of DNAse (Roche, 104 159), it was stirred for 30 minutes at room temperature. The supernatant was collected and spinned down for 5 min at 1500 rpm (in case of the cells being fussy, starter medium (L15, 10% FCS, 100 mM 2-mercaptoethanol) or complete MLC should be used). The cells obtained by this precedure were washed with F15+10% FCS twice, spinned down, resuspended in F15+10% FCS and counted. The cells were grown on 6-well plates, with a dilution of 5×10⁵ cells/well giving the best results. After passing the cells 10 times and freezing the stocks, the cell line was ready to use.

### EXAMPLE 3

### Screening for Antifungal Agents

Screening for antifungal agents capable of inhibiting the interaction between GT and Bak can be performed by means of the methylcholanthrene-induced cell line MC.Fas^{-/-} (date of deposition at DSMZ: 4 May 2006) stained with the vital dye Neutral Red. The MC.Fas^{-/-} cells (date of deposition at DSMZ: 4 May 2006) are allowed to adhere to the plates and are subsequently incubated with GT in the presence or absence of a compound potentially inhibiting GT. After an appropriate incubation time, the wells are washed to remove non-adherent cells. Neutral Red is released from the remaining cells by acetic acid/ethanol. Cell loss equates with cell death resulting from GT-induced pro-apoptotic processes, and is quantified automatically by loss of absorbance at 540 nm using a microplate reader.

### RESULTS

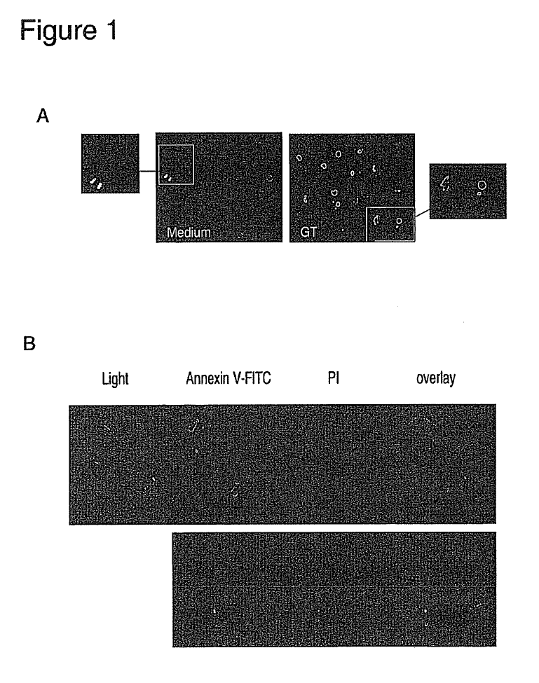

As shown in FIG. 1, 1 μM GT induced nuclear fragmentation in the majority of wild-type (wt) MEFs. Furthermore, ˜50% of these cells were apoptotic, i.e. had phosphatidylserine (PS) exposed (annexin-V staining) without plasma membrane disruption while the rest of the cells already showed secondary necrosis (loss of membrane integrity as shown by propidium iodide (PI) staining) (FIG. 1; and [Pardo, 2004]). Thus, 1 μM GT was used in all subsequent experiments.

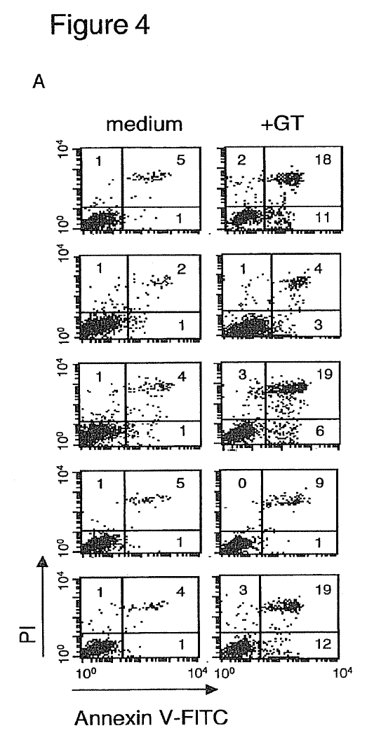

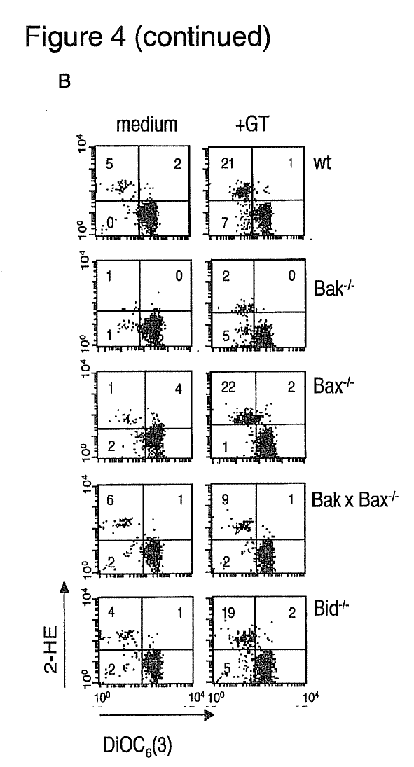

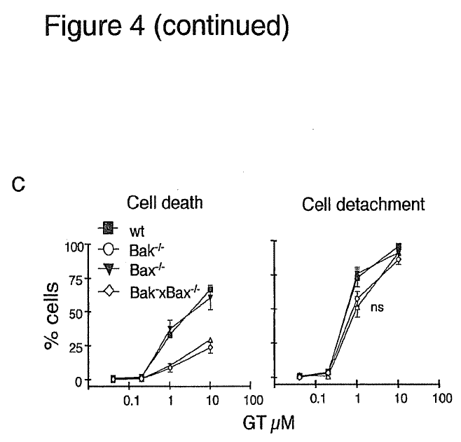

We next compared GT-induced PS exposure and PI staining between wt and knock-out (−/−) MEFs. In addition, we studied the impact of GT on the mitochondrial membrane potential (Δψₘ) and the production of reactive oxygen species (ROS) in these cells. As shown in FIG. 4A, GT significantly increased the number of annexin-V/PI positive cells in wt, Bak^{-/-} and Bid^{-/-} MEFs as compared to mock treated cells, while Bak^{-/-} and Bak^{-/-}×Bax^{-/-} MEFs did not. Similarly, GT treatment led to a significant reduction in the Δψₘ and a parallel increase in ROS production in wt, Bax^{-/-} and Bid^{-/-}, but not in Bak^{-/-} and Bak^{-/-}×Bax^{-/-} MEFs (FIG. 4B). These data suggest that in MEFs, Bak, but not Bax, is critical for GT-induced loss of plasma membrane integrity and the mitochondrial membrane potential. Moreover, Bid, which is known to activate Bak and Bax during apoptosis [Wei, 2001], seems to be dispensable for these processes. In support of a key role of Bak in GT-mediated cell death, we found that trypan blue exclusion was significantly reduced only in Bak^{-/-} and Bak^{-/-}×Bax^{-/-}, but not in wt or Bax^{-/-} MEFs (FIG. 4C). In contrast, GT-induced loss of cell adherence [Mullbacher, 1985] was independent of Bax or Bak as it was only marginally, if at all, diminished in both Bax- and/or Bak-deficient MEFs (FIG. 4C).

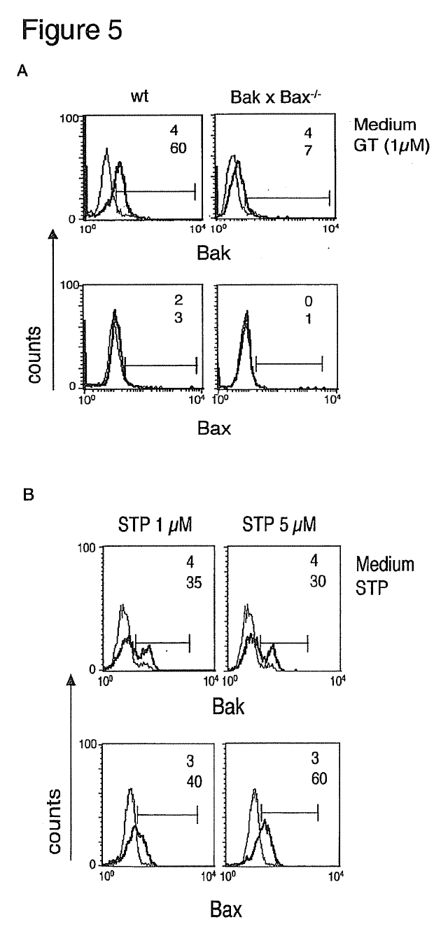

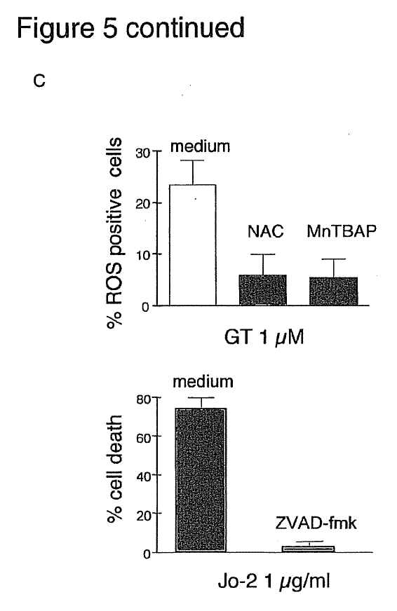

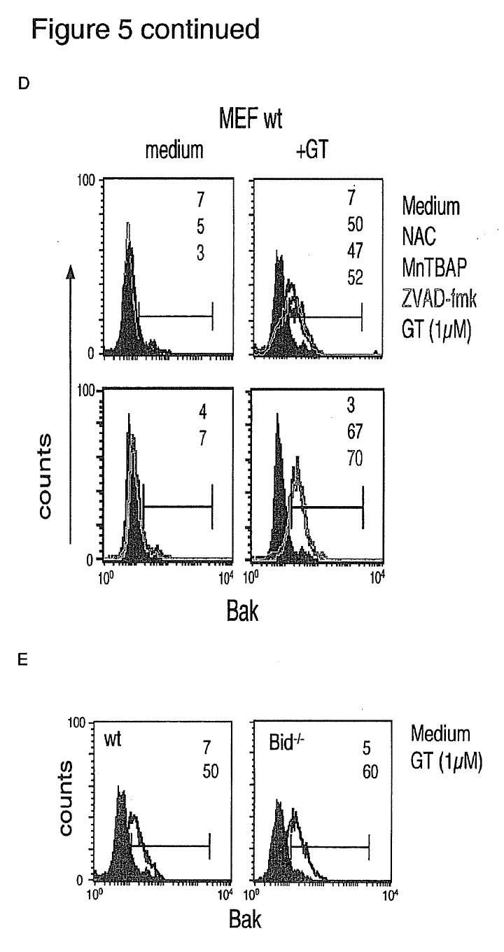

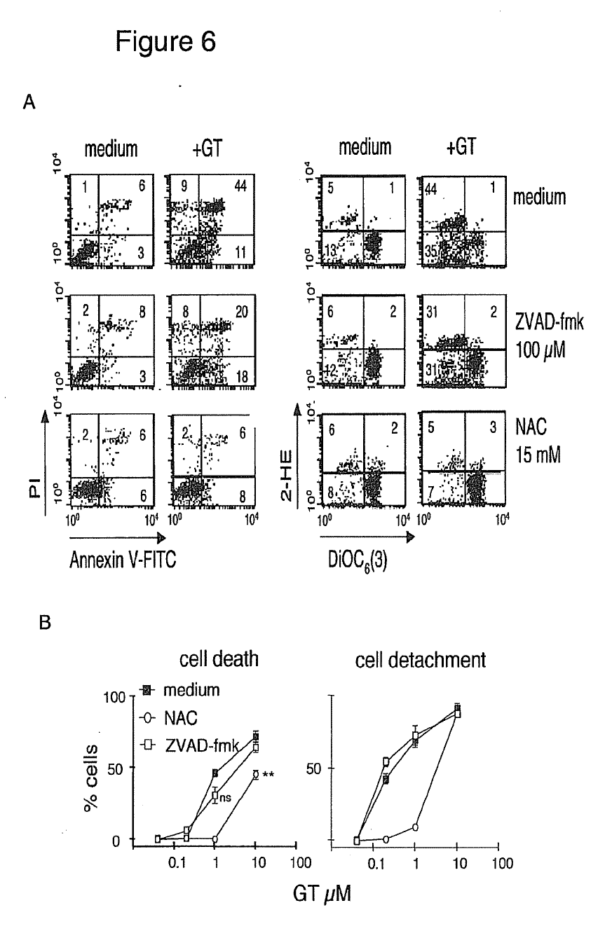

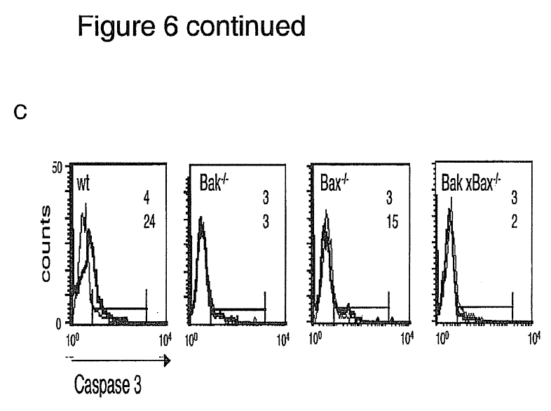

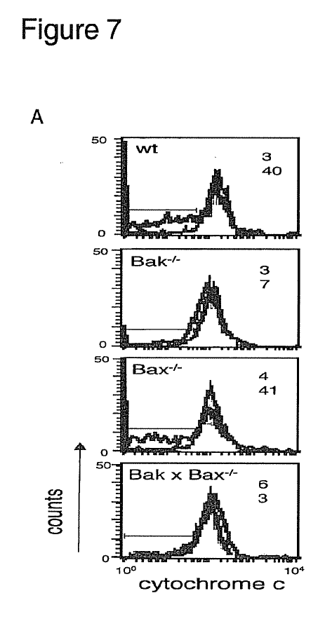

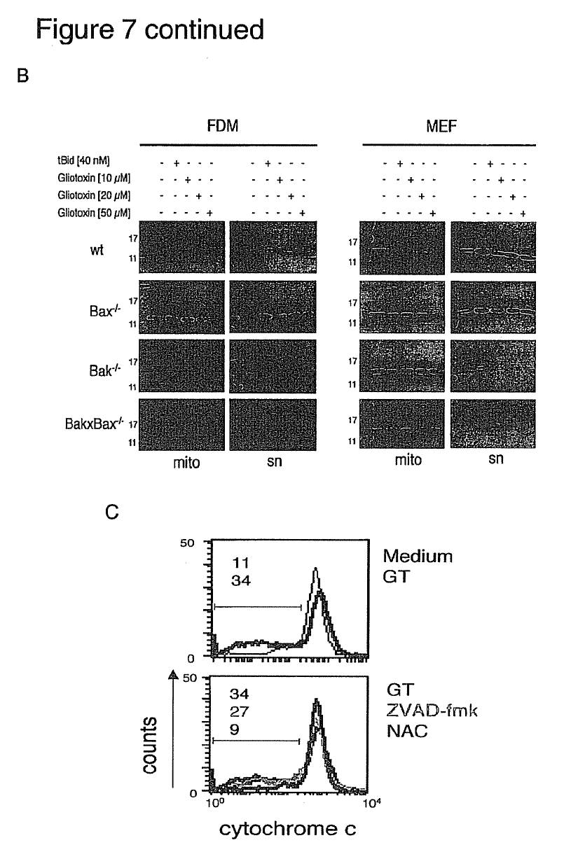

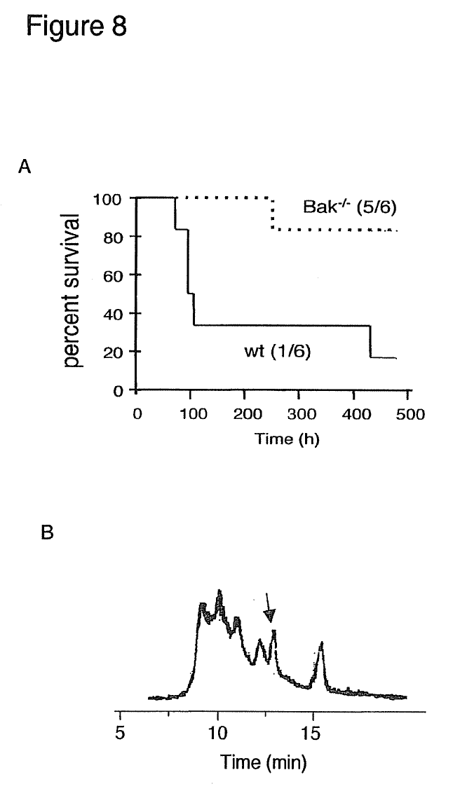

Upon their activation, Bak and Bax undergo conformational changes leading to the exposure of their N-terminal domains [Gao, 2000; Denisov, 2003]. To test if this process also occurs during GT treatment, wt and Bak^{-/-}×Bax^{-/-} MEFs were incubated with GT and subsequently analysed by FACS with conformation-specific antibodies against the N-termini of Bak or Bax. As shown in FIG. 5A, GT was able to readily induce N-terminal epitope exposure in Bak, but not in Bax. As expected, no (Bax) or only background (Bak) staining was seen with these antibodies in GT-activated Bak^{-/-}×Bax^{-/-} MEFs. There was no inherent failure of Bax to undergo a N-terminal conformational change in MEFs, as their treatment with the apoptosis inducing drug staurosporine led to the expected N-terminal opening of Bax (FIG. 5B). Thus, while staurosporine activates both Bax and Bak, GT appears to activate only Bak in MEFs.

To determine the order of events during Bak activation, ROS production and caspase activation, we incubated GT-treated MEFs with the antioxidants N-acetylcysteine (NAC) or the manganese porphirin Mn(III) tetrakis(4-benzoic acid)porphirin chloride (MnTBAP) [Day, 1997; Faulkner, 1994] or the pan-caspase inhibitor, Z-VAD-fmk [Pardo, 2004]. NAC and MnTBAP were both effective as antioxidants as they significantly reduced GT-induced ROS production in MEFs (FIG. 5). Moreover, Z-VAD.fmk blocked apoptosis induced by the anti-Fas antibody Jo-2 (FIG. 5C). GT-induced N-terminal opening of Bak was unaffected by any of the three inhibitors (FIG. 5) indicating that ROS production and caspase activation occur downstream of Bak activation. Furthermore, since similar activation of Bak was seen in GT-treated wt- and Bid^{-/-} MEFs, GT-mediated conformational change of Bak is also independent of Bid (FIG. 5).

Although the association of ROS generation with GT- or CTL-mediated apoptosis is well documented [Kweon, 2003; Pardo, 2004], the contribution of ROS to cell death is still controversial [MacDonald, 1999; Barry, 2000; Heibein, 2000; Danial, 2004]. We therefore tested the effect of NAC on GT-induced plasma and mitochondrial membrane integrity and cell death. As seen in FIG. 6, the addition of NAC to wt MEFs, at a concentration known to inhibit ROS generation (FIG. 6 and [Pardo, 2004]), abrogated GT-induced PS exposure and plasma membrane permeability as well as the reduction of the mitochondrial Δψₘ. Moreover, at 1 μM GT, NAC totally inhibited cell death (according to absence of trypan blue staining) and cell detachment (FIG. 6). These data reveal that ROS generation is crucial for GT-induced changes of the mitochondrial Δψₘ and cell death.

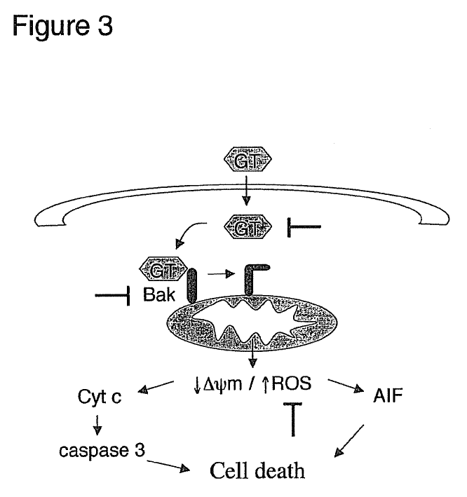

To determine the role of caspases in the GT-induced reduction of the mitochondrial Δψₘ and apoptosis, we tested whether GT could induce cell death and changes in mitochondrial Δψₘ in the presence of the broad spectrum caspase inhibitor Z-VAD.fmk. 100 μM Z-VAD.fmk did not prevent loss of the mitochondrial Δψₘ in GT-treated cells indicating that caspase activation was not needed for this event. However, Z-VAD.fmk reduced GT-induced annexin-V/PI staining (FIG. 6) and cell death (FIG. 6). To test if the major downstream caspase, caspase-3 was involved in these processes, and if activation of this caspase was dependent on Bak, we performed a FACS analysis of wt and −/− MEFs using an anti-caspase-3 antibody specific for the processed active form of caspase-3. As shown in FIG. 3C, caspase-3 was significantly activated in GT-treated wt and Bax^{-/-} but not in Bak^{-/-} and Bak^{-/-}×Bax^{-/-} MEFs.

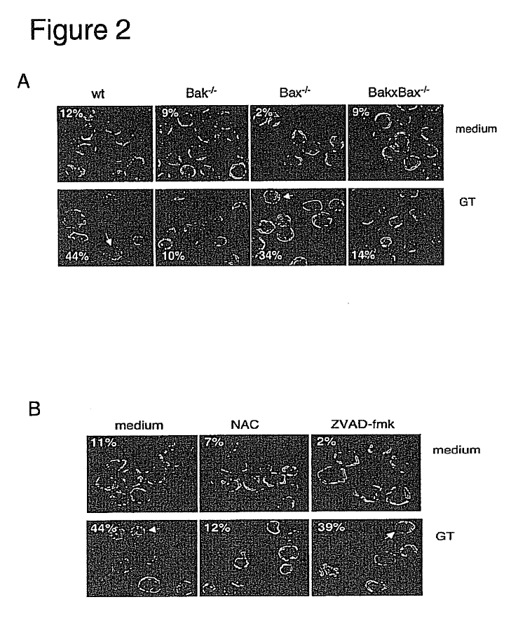

The production of ROS and the reduction of mitochondrial Δψₘ in response to GT is indicative of increased mitochondrial membrane permeability, leading to the release of apoptogenic factors such as cytochrome c and apoptosis inducing factor (AIF). While cytochrome c activates caspase-3 via the apoptosome, AIF translocates to the nucleus and contributes to DNA fragmentation in a caspase-independent manner [Susin, 1999; Pardo, 2001]. The release of these factors from mitochondria is absolutely dependent on Bax and Bak since cells deficient for both Bax and Bak retain mitochondrial integrity despite exposure to a range of apoptotic stimuli [Shimizu, 1999; Wei, 2001; Wei, 2000]. To test if GT-induced cytochrome c release selectively required Bak, wt and −/− MEF cell lines were incubated with GT and mitochondrial cytochrome c was quantitatively measured by FACS analysis (FIG. 7). In addition, AIF release was monitored by anti-AIF immunofluorescence (FIG. 2). As shown in FIG. 7, mitochondrial cytochrome c was reduced in GT-treated wt and Bax−/− MEFs but retained in Bak^{-/-} or Bak^{-/-}×Bax^{-/-} MEFs. In addition, a high portion of GT-treated wt and Bax^{-/-} MEFs displayed cytosolic localization and nuclear translocation of AIF whereas Bak^{-/-} and Bak^{-/-}×Bax^{-/-} MEFs retained most of the AIF in the mitochondria (FIG. 2A). These data confirm that GT selectively interacts with Bak and elicits increased mitochondrial membrane permeability, i.e cytochrome c and AIF release. To test if GT could directly act on mitochondrial Bak without the requirement of any cytosolic factors, we performed GT-induced cytochrome c release on isolated mitochondrial from wt and ko MEFs and FDMs. As shown in FIG. 7, GT caused cytochrome c release from isolated mitochondria in a dose-dependent manner. This release was as efficient as that induced by recombinant tBid, a known inducer of mitochondrial membrane permeability via Bak/Bax [Wei, 2000; Shimizu, 1999; Wei, 2001; Wang, 2001] and significantly greater than the background release of cytochrome observed in mock-treated mitochondrial preparations. Strikingly, while mitochondria from Bax^{-/-} showed similar GT-induced cytochrome c release as wt mitochondria, such was absent in mitochondria from Bak^{-/-} or Bak^{-/-}×Bax^{-/-} mitochondria, irrespective of whether they were derived from MEFs or FDMs. These data suggest that GT does not need cytosolic factors such as tBid or caspases to elicit cytochrome c release but may directly act on mitochondrial Bak or some unknown mitochondrial protein that activates Bak with a similar potency as tBid. Consistent with this idea GT-induced cytochrome c and AIF release was not blocked by Z-VAD (FIG. 7 and FIG. 2B).

Interestingly, both cytochrome c and AIF release from mitochondria was greatly diminished by NAC (FIG. 7 and FIG. 2B) suggesting either that GT-induced Bak activation somehow triggers ROS production that is crucial for effective mitochondrial membrane pore formation or that NAC has some other mitochondrial membrane stabilizing activity.

Finally, to determine whether the selective activation of Bak during GT-mediated cell death is of pathophysiological significance, we monitored the mortality of immuno-suppressed (hydrocortisone-treated) wild type (C57BL/6) and Bak ko (Bak^{-/-}) mice subsequently infected with a GT producing A. fumigatus strain (FIG. 8). FIG. 8 shows that 5 out of 6 wt mice died within two weeks of intranasal infection but only 1 out of 6 Bak^{-/-} mice succumbed over the same time period. These in vivo data correlate with our in vitro findings and show for the first time that Bak is a host susceptibility factor for A. fumigatus virulence in mice, probably due to its direct activation by GT. In conclusion, the present study suggests the sequence of intracellular events during GT-induced apoptosis, a model which is schematically represented in FIG. 3 and defines Bak as a promising target for therapeutic intervention of A. fumigatus infections in man. Further experiments are required to determine if GT could act as a tBid-like BH3-only mimetic to directly activate Bak or if other mitochondrial membrane proteins/factors are required for this activation. Moreover, it will be important to understand the mechanism by which GT-activated Bak produces ROS and how this in turn triggers cytochrome c release, caspase activation and cell death.

### Discussion

Although GT has long been proposed to constitute a virulence factor in IA [Mullbacher, 1984; Eichner 1984], most probably by suppressing immune responses via induction of mammalian cell apoptosis [Waring, 1988; Sutton, 1994], the molecular mechanism(s) underlying the putative in vitro and in vivo processes has not been elucidated. Here we present evidence that Bak, but not Bax is a key host factor in GT-mediated cell death in vitro. Experiments, using mouse embryo fibroblasts (MEFs) and IL-3-(factor) dependent mouse monocytes (FDMs) deficient in the putative pore-forming Bcl-2 family members Bak and/or Bax or their activator Bid [Wei, 2000], and isolated mitochondria from these cells revealed that GT-mediated activation of Bak occurs independently of Bid or other cytosolic factors. Once activated, Bak triggers the generation of ROS, which is crucial for effective mitochondrial membrane pore formation, including the release of cytochrome c and AIF, and ultimate cell death. The additional finding that the virulence of GT-producing A. fumigatus was significantly decreased in Bak−/− over wt mice strongly implicates GT as an important modulator in mammalian host defence and that Bak is a prominent host susceptibility factor.

In healthy cells, Bak is constitutively bound to mitochondria and kept inactive by interaction with either pro-survival Bcl-2 family members, such as Bcl-XL, Bcl-2 and Mcl-1 [Willis, 2005; Sattler, 1997; Cuconati, 2003; Ekert et al., submitted], VDAC2 [Cheng; 2003], or both. The BH3-only proteins tBid and Bim can directly activate Bak in isolated mitochondria [Wei, 2000; Kuwana, 2005], but the physiological relevance of this process is unclear. Alternatively, after activation in apoptotic cells, BH3-only proteins bind to the pro-survival factors with high affinity [Chen, 2005] and thereby displace Bak for conformational change, oligomerization and pore formation. This has recently been shown for UV-induced apoptosis where NOXA together with an as yet unknown BH3-only protein liberated Bak from Mcl-1 and Bcl-XL, respectively [Willis, 2005].

Without wishing to be bound by theory, one mechanism by which GT may activate Bak is by breaking up inhibitory complexes between Bak and Bcl-2-like pro-survival factors or VDAC2 on the mitochondrial membrane. This could be by forming transient disulphide bonds between the reactive disulphide bond in GT and individual cysteine residues in Bak or its binding partners, leading to the release of active Bak. Our data suggest that BH3-only proteins may not be involved in the mode of action of GT, for the following reasons. Firstly, we show here that GT activates Bak, cytochrome c release and apoptosis independent of Bid, one of the most prominent candidates for Bak activation [Wei, 2000]. Secondly, GT-mediated and Bak facilitated mitochondrial membrane disruption could not be blocked by the general inhibitor (Z-VAD.fmk) for caspases, known to be critical in processing Bid into active tBid. Thirdly and most importantly, GT can induce cytochrome c release on isolated mitochondria without the participation of cytosolic factors and protein synthesis. Although low amounts of some BH3-only proteins such as for example Bim can be constitutively found on mitochondria from B and T cells [Gomez-Bougie, 2005; Zhu, 2004], as well as fibroblasts, most BH3-only proteins are cytosolic or attached to the cytoskeleton [Huang, 2000; Puthalakath, 1999]. Furthermore some of them are synthesized de novo only after apoptotic stimuli via p53 activation, such as NOXA or PUMA [Oda, 2000; Vousden, 2005]. Thus, our data obtained with isolated mitochondria favor the interpretation that GT-facilitated activation of Bak occurs by direct interaction with anti-apoptotic Bcl-2 family members or other mitochondrial membrane associated constituents. The former possibility is supported by the observation that protection against GT-mediated monocyte apoptosis by agonists of nerve growth factor receptors is associated with upregulation of Bcl-2 and Bcl-xL [la Sala, 2000].

A compelling aspect of our study is, that GT specifically acts through Bak and not Bax. Although both proteins are supposed to exert the same pore-forming activity on mitochondria [Kuwana, 2005] they are activated differently. In fact, there is increasing evidence for selective Bax- or Bak-specific apoptosis depending on the cell type and the apoptotic stimuli [Wendt, 2005; Lindenboim, 2005]. In contrast to Bak, Bax is a cytosolic or loosely mitochondria-attached protein, kept inactive by occluding its C-terminal mitochondrial targeting sequence into the hydrophobic pocket [Suzuki, 2000; Schinzel, 2004] and binding additional inhibitory proteins [Nomura, 2003; Sawada 2004; Guo, 2003], but not by interacting with Bcl-2-like survival factors [Willis, 2005; and own unpublished results]. Apart from a possible hit-and-run activation mechanism by tBid and Bim [Kuwana; 2005], as also suggested for Bak activation, it is unknown how Bax is induced for C-terminal unleashment, mitochondrial translocation, oligomerization and pore formation. In this respect, GT may be unable to interact with Bax or any of its inhibitory components. Moreover, an interaction of GT with Bcl-2 or Bcl-xL would not affect Bax because it is not sequestered by these proteins in healthy cells. This would explain why GT induces conformational activation of Bak, but not of Bax.

In consideration of the findings that the mitochondrial protein VDAC2 associates with and inhibits Bak in healthy mitochondria [Cheng; 2003] and that in monocytes, Bak but not Bax is part of the VDAC channel (own unpublished results), we have concluded that VDAC2 may be involved in GT-mediated cell death. VDAC2 is one of three mammalian isoforms of VDAC proteins (VDAC1, VDAC2 and VDAC3), which constitute the major pathway for metabolic exchange across the outer mitochondrial membrane [Sampson, 1997; Wu, 1999; Xu, 1999]. Together with cyclophilin D and adenine nucleotide transporter (ANT), VDAC forms the mitochondrial permeability transition pore (MTPT), involved in cell apoptosis and/or necrosis [Crompton, 1999; Zheng, 2004]. How the function of MPTP is regulated by members of the BH3 family is still highly controversial [Shimizu, 1999; Marzo, 1998; Vander Heiden, 1999]. One could postulate that GT somehow modulates the VDAC complex, leading to the liberation of Bak, a subsequent increase of mitochondrial membrane permeability and hence a Bak-dependent cytochrome c release and cell death. The contribution of the MPTP in the latter process is further supported by the findings that GT-induced apoptosis of activated hepatic stellate cells is associated with a specific thiol redox-dependent interaction with MPTP component ANT [Orr, 2004] and that cyclosporin A, an inhibitor of cyclophilin D and mitochondrial pore opening [Crompton, 1988], affected mitocondrial depolarization and ROS production [Kweon, 2003]. Most notably, the data suggested that oxidative cross-linking of two matrix-facing cysteine residues on the ANT (Cys₅₆ and Cys₁₅₉) plays a key role in regulating the MPTP [Halestrap, 2002].

Our data further show that GT-induced production of ROS is mandatory for cell death. The sequence of events leading to ROS production by GT was revealed by analysing the various pro-apoptotic processes in the presence of inhibitors for ROS and for caspases, including NAG, MnTBAP and Z-VAD-fmk, respectively (FIG. 3). Accordingly, activation of Bak precedes the generation of ROS, which then facilitate the release of cytochrome c and AIF from mitochondria, leading to caspase activation as well as mitochondria- and caspase-independent events to mediate cell death. As to the source of ROS, it is possible that they are generated from a perturbance of mitochondrial respiration due to Bak-mediated pore formation and/or activation of MPTP. Why ROS are, at least partially, required for cytochrome c release is unclear. Perhaps they facilitate the further recruitment of cytochrome c from internal cristae pools as it was proposed for the action of tBid [Scorrano, 2002] or contribute to the dissociation of cytochrome c from the mitochondrial inner membrane by cardiolipin peroxidation [Petrosillo, 2001; Orrenius, 2005].

The relevance of GT-mediated apoptosis for the parasitized vertebrate host, was verified by comparing the course of A. fumigatus infection in wt and Bak_{-/-} mice. The significantly decreased virulence of the pathogen observed in Bak_{-/-} as compared to wt mice, as revealed by the differential kinetics of mortality, strongly implicates that GT is released during A. fumigatus infection and induces apoptosis in multiple target cells via Bak activation. This process(es) subsequently leads to both, an accelerated colonization of target organs by breaching physical barriers, such as lung and renal epithelial cells, and establishes an immunsuppressed state of the host. Our results have shown that low levels of pulmonary GT observed with an A. fumigatus mutant defective in LaeA, a global regulator of secondary metabolism, is associated with impaired virulence of the pathogen. Furthermore, by employing a recently generated glip gene knockout mutant of A. fumigatus lacking GT, we found that this mutant is much less virulent in mice than the wild type strain and that cell culture supernatants were unable to induce cell death (own unpublished results).

Based on the sequence of intracellular events occurring during GT-induced apoptosis (see scheme, FIG. 3), we conclude that GT is a critical virulence factor in A. fumigatus. This is supported by the fact that GT is one of the most abundant secondary metabolites produced by the fungus [Taylor, 1971] and that Bak_{-/-} mice are more resistant to infection by A. fumigatus. Our recent findings that GT is the predominant apoptogenic factor of A. fumigatus (own unpublished results) further support this contention. The distinct potential of GT to activate Bak, but not Bax may be of relevance for the development of an anti-IA drug that selectively blocks cell death pathways via Bak and, at the same time, spares the residual proapototic proteins relevant for the control of the pathogen by the host's immune system. Further experiments are required to determine if GT can act as a tBid-like BH3-only mimetic. to directly activate Bak or if other mitochondrial membrane proteins/factors are required for this activation. Understanding of the mechanism by which GT-activated Bak, produces ROS and how this in turn triggers cytochrome c and AIF release, caspase activation and cell death will be essential for developing therapeutics targeting the apoptotic response.

### LIST OF REFERENCES

Adachi, M. S., S. Suematsu, T. Kondo, J. Ogasawara, T. Tanaka, N. Yoshida, S. Nagata (1995), Nature Genetics, 11, 294-300.
Barry, M., J. A. Heibein, M. J. Pinkoski, S.-F. Lee, R. W. Moyer, D. R. Green, R. C. Bleackley (2000), Mol Cell Biol, 20, 3781-3794.
Belkacemi, L., R. C. Barton, V. Hopwood, E. G. Evans (1999), Med Mycol, 37, 227-233.
Beste, G., F. S. Schmidt, T. Stibora, A. Skerra (1999), Proc Natl Acad Sci USA, 96, 1898-1903.
Bok, J. W., S. A. Balajee, K. A. Marr, D. Andes, K. F. Nielsen, J. C. Frisvad, N. P. Keller (2005), Eukaryot Cell, 4, 1574-1582.
Chen, L., S. N. Willis, A. Wei, B. J. Smith, J. I. Fletcher, M. G. Hinds, P. M. Colman, C. L. Day, J. M. Adams, D. C. Huang (2005), Mol Cell, 17, 393-403.
Cheng, E. H., T. V. Sheiko, J. K. Fisher, W. J. Craigen, S. J. Korsmeyer (2003), Science, 301, 513-517.
Crompton, M., H. Ellinger, A. Costi (1988), Biochem J, 255, 357-360.
Crompton, M. (1999), Biochem J, 341 (Pt 2), 233-249.
Cuconati, A., C. Mukherjee, D. Perez, E. White (2003), Genes Dev, 17, 2922-2932.
Danial, N. N., S. J. Korsmeyer (2004), Cell, 116, 205-219.
Day, B. J., I. Fridovich, J. D. Crapo (1997), Arch Biochem Biophys, 347, 256-262.
Denisov, A. Y., M. S. Madiraju, G. Chen, A. Khadir, P. Beauparlant, G. Attardo, G. C. Shore, K. Gehring (2003), J Biol Chem, 278, 21124-21128.
Eichner, R. D., A. Mullbacher (1984), Aust J Exp Biol Med Sci, 62 (Pt 4), 479-484.
Eichner, R. D., P. Waring, A. M. Geue, A. W. Braithwaite, A. Mullbacher (1988), J Biol Chem, 263, 3772-3777.
Ekert, P. G., S. H. Read, J. Silke, V. S. Marsden, H. Kaufmann, C. J. Hawkins, R. Geri, S. Kumar, D. L. Vaux (2004), J Cell Biol, 165, 835-842.
Faulkner, K. M., R. D. Stevens, I. Fridovich (1994), Arch Biochem Biophys, 310, 341-346.
Gao, G., Q. P. Dou (2000), J Cell Biochem, 80, 53-72.
Gomez-Bougie, P., R. Bataille, M. Amiot (2005), Eur J Immunol, 35, 971-976.
Guo, B., D. Zhai, E. Cabezas, K. Welsh, S. Nouraini, A. C. Satterthwait, J. C. Reed (2003), Nature, 423, 456-461.
Halestrap, A. P., G. P. McStay, S. J. Clarke (2002), Biochimie, 84, 153-166.
Heibein, J. A., I. S. Goping, M. Barry, M. J. Pinkoski, G. C. Shore, D. R. Green, R. C. Bleackley (2000), J Exp Med, 192, 1391-1402.
Huang, D. C., A. Strasser (2000), Cell, 103, 839-842.
Kuwana, T., L. Bouchier-Hayes, J. E. Chipuk, C. Bonzon, B. A. Sullivan, D. R. Green, D. D. Newmeyer (2005), Mol Cell, 17, 525-535.
Kweon, Y. O., Y. H. Paik, B. Schnabl, T. Qian, J. J. Lemasters, D. A. Brenner (2003), J Hepatol, 39, 38-46.
la Sala, A., S. Corinti, M. Federici, H. U. Saragovi, G. Girolomoni (2000), J Leukoc Biol, 68, 104-110.
Latge, J. P. (1999), Clin Microbiol Rev, 12, 310-350.
Latge, J. P. (2001), Trends Microbiol, 9, 382-389.
Lewis, R. E., N. P. Wiederhold, J. Chi, X. Y. Han, K. V. Komanduri, D. P. Kontoyiannis, R. A. Prince (2005), Infect Immun, 73, 635-637.
Lindenboim, L., S. Kringel, T. Braun, C. Borner, R. Stein (2005), Cell Death Differ, 12, 713-723.
Lindsten, T., A. J. Ross, A. King, W.-X. Zong, J. C. Rathmell, H. A. Shiels, E. Ulrich, K. G. Waymire, P. Mahar, K. Frauwirth, Y. Chen, M. Wei, V. M. Eng, D. M. Adelman, M. C. Simon, A. Ma, J. A. Golden, G. Evan, S. J. Korsmeyer, G. R. MacGregor, C. B. Thompson (2000), Mol Cell, 6, 1389-1399.
MacDonald, G., L. Shi, C. Vande Velde, J. Lieberman, A. H. Greenberg (1999), J Exp Med, 189, 131-144.
Marzo, I., C. Brenner, G. Kroemer (1998), Biomed Pharmacother, 52, 248-251.
Mullbacher, A., R. D. Eichner (1984), Proc Natl Acad Sci USA, 81, 3835-3837.
Mullbacher, A., P. Waring, R. D. Eichner (1985), J Gen Microbiol, 131, 1251-1258.
Nomura, T., H. Mimata, Y. Takeuchi, H. Yamamoto, E. Miyamoto, Y. Nomura (2003), Urol Res, 31, 37-44.
Oda, E., R. Ohki, H. Murasawa, J. Nemoto, T. Shibue, T. Yamashita, T. Tokino, T. Taniguchi, N. Tanaka (2000), Science, 288, 1053-1058.
Orr, J. G., V. Leel, G. A. Cameron, C. J. Marek, E. L. Haughton, L. J. Elrick, J. E. Trim, G. M. Hawksworth, A. P. Halestrap, M. C. Wright (2004), Hepatology, 40, 232-242.
Orrenius, S., B. Zhivotovsky (2005), Nat Chem Biol, 1, 188-189.
Pardo, J., P. Perez-Galan, S. Gamen, I. Marzo, I. Monleon, A. A. Kaspar, S. A. Susin, G. Kroemer, A. M. Krensky, J. Naval, A. Anel (2001), J Immunol, 167, 1222-1229.
Pardo, J., A. Bosque, R. Brehm, R. Wallich, J. Naval, A. Mullbacher, A. Anel, M. M. Simon (2004), J Cell Biol, 167, 457-468.
Petrosillo, G., F. M. Ruggiero, M. Pistolese, G. Paradies (2001), FEBS Lett, 509, 435-438.
Puthalakath, H., D. C. Huang, L. A. O'Reilly, S. M. King, A. Strasser (1999), Mol Cell, 3, 287-296.
Sampson, M. J., R. S. Lovell, W. J. Craigen (1997), J Biol Chem, 272, 18966-18973.
Sattler, M., H. Liang, D. Nettesheim, R. P. Meadows, J. E. Harlan, M. Eberstadt, H. S. Yoon, S. B. Shuker, B. S. Chang, A. J. Minn, C. B. Thompson, S. W. Fesik (1997), Science, 275, 983-986.
Sawada, H., R. Kohno, T. Kihara, Y. Izumi, N. Sakka, M. Ibi, M. Nakanishi, T. Nakamizo, K. Yamakawa, H. Shibasaki, N. Yamamoto, A. Akaike, M. Inden, Y. Kitamura, T. Taniguchi, S. Shimohama (2004), J Biol Chem, 279, 10710-10719.
Schinzel, A., T. Kaufmann, C. Borner (2004), Biochim Biophys Acta, 1644, 95-105.
Schlehuber, S., A. Skerra (2001), Biol Chem, 382, 1335-1342.
Scorrano, L., M. Ashiya, K. Buttle, S. Weiler, S. A. Oakes, C. A. Mannella, S. J. Korsmeyer (2002), Dev Cell, 2, 55-67.
Shimizu, S., M. Narita, Y. Tsujimoto (1999), Nature, 399, 483-487.
Skerra, A. (2001), Rev Mol Biotechnol, 74, 257-275.
Stanzani, M., E. Orciuolo, R. Lewis, D. P. Kontoyiannis, S. L. R. Martins, L. S. St. John, K. V. Komanduri (2005), Blood, 105, 2258-2265.
Suen, Y. K., K. P. Fung, C. Y. Lee, S. K. Kong (2001), Free Radic Res, 35, 1-10.
Susin, S. A., H. K. Lorenzo, N. Zamzami, I. Marzo, B. E. Snow, G. M. Brothers, J. Mangion, E. Jacotot, P. Costantini, M. Loeffler, N. Larochette, D. R. Goodlett, R. Aebersold, D. P. Siderovski, J. M. Penninger, G. Kroemer (1999), Nature, 397, 441-446.
Sutton, P., N. R. Newcombe, P. Waring, A. Mullbacher (1994), Infect Immun, 62, 1192-1198.
Sutton, P., P. Waring, A. Mullbacher (1996), Immunol Cell Biol, 74, 318-322.
Suzuki, M., R. J. Youle, N. Tjandra (2000), Cell, 103, 645-654.
Tang, C. M., J. Cohen, T. Krausz, S. Van Noorden, D. W. Holden (1993), Infect Immun, 61, 1650-1656.
Taylor, A., In Microbial Toxins. S. Kadis, A. Ciegler, S. J. Ajl, editors. Academic Press, New York, 1971, 337-376.
van den Broek, M. E., D. Kägi, F. Ossendorp, R. Toes, S. Vamvakas, W. K. Lutz, C. J. M. Melief, R. M. Zinkernagel, H. Hengartner (1996), J Exp Med, 184, 1781-1790.
Vander Heiden, M. G., N. S. Chandel, P. T. Schumacker, C. B. Thompson (1999), Mol Cell, 3, 159-167.
Vousden, K. H. (2005), Science, 309, 1685-1686.
Wang, G. Q., B. R. Gastman, E. Wieckowski, L. A. Goldstein, A. Gambotto, T. H. Kim, B. Fang, A. Rabinovitz, X. M. Yin, H. Rabinowich (2001), J Biol Chem, 276, 34307-34317.
Waring, P., R. D. Eichner, A. Mullbacher, A. Sjaarda (1988), J Biol Chem, 263, 18493-18499.
Wasilenko, S. T., L. Banadyga, D. Bond, M. Barry (2005), J Virol, 79, 14031-14043.
Waterhouse, N. J., J. A. Trapani (2003), Cell Death Differ, 10, 853-855.
Wei, M. C., T. Lindsten, V. K. Mootha, S. Weiler, A. Gross, M. Ashiya, C. B. Thompson, S. J. Korsmeyer (2000), Genes Dev, 14, 2060-2071.
Wei, M. C., W. X. Zong, E. H. Cheng, T. Lindsten, V. Panoutsakopoulou, A. J. Ross, K. A. Roth, G. R. MacGregor, C. B. Thompson, S. J. Korsmeyer (2001), Science, 292, 727-730.
Wendt, J., C. von Haefen, P. Hemmati, C. Belka, B. Dorken, P. T. Daniel (2005), Oncogene, 24, 4052-4064.
Willis, S. N., L. Chen, G. Dewson, A. Wei, E. Naik, J. I. Fletcher, J. M. Adams, D. C. Huang (2005), Genes Dev, 19, 1294-1305.
Wu, S., M. J. Sampson, W. K. Decker, W. J. Craigen (1999), Biochim Biophys Acta, 1452, 68-78.
Xu, X., W. Decker, M. J. Sampson, W. J. Craigen, M. Colombini (1999), J Membr Biol, 170, 89-102.
Zheng, Y., Y. Shi, C. Tian, C. Jiang, H. Jin, J. Chen, A. Almasan, H. Tang, Q. Chen (2004), Oncogene, 23, 1239-1247.
Zhou, X., A. Zhao, G. Goping, P. Hirszel (2000), Toxicol Sci, 54, 194-202.
Zhu, Y., B. J. Swanson, M. Wang, D. A. Hildeman, B. C. Schaefer, X. Liu, H. Suzuki, K. Mihara, J. Kappler, P. Marrack (2004), Proc Natl Acad Sci USA, 101, 7681-7686.

1. (canceled)

2. The method of claim 22 wherein the inhibitor suppresses GT-mediated pro-apoptotic and/or apoptotic processes.

3. The method of claim 22 wherein the inhibitor selectively suppresses apoptotic activation of Bak.

4. The method of 22 wherein GT-mediated activation of Bak proceeds via modulation of VDAC2.

5. The method of claim 22 wherein the inhibitor binds to GT, VDAC2 and/or to Bak.

6. The method of claim 22 wherein the inhibitor is a polypeptide molecule which binds to GT, VDAC2 and/or to Bak.

7. The method of claim 22 wherein the inhibitor is selected from antibodies, anticalins, scaffold molecules, aptamers, spiegelmers and chemical compounds.

8. The method of claim 22 wherein the inhibitor is a nucleic acid molecule which inhibits Bak expression.

9. The method of claim 8 wherein the inhibitor is selected from antisense molecules, ribozymes and nucleic molecules capable of RNA interference.

10. The method of claim 22 for the prevention and/or treatment of infections with Aspergillus species and/or Candida species.

11. The method of claim 22 in human or veterinary medicine.

12. The method of claim 22 in immuno-compromised or immuno-suppressed patients.

13. A method for identifying antifungal agents comprising determining whether a compound is capable of inhibiting the interaction between GT and Bak.

14. The method of claim 13 comprising determining whether a compound suppresses pro-apoptotic and/or apoptotic processes mediated by GT.

15. The method of claim 14 wherein the determination is carried out in Fas-negative cells.

16. The method of claim 15 wherein the Fas-negative cells are mammalian and/or tumor cells.

17. The method of claim 15 comprising determination of cell adherence, wherein loss of cell-adherence equates with cell death.

18. The method of claim 15 comprising determination of cell survival, wherein loss of the vital dye neutral red equates with cell death.

19. A test system for identifying antifungal agents comprising a Fas-negative cell.

20. The test system of claim 19, wherein the Fas-negative cell is a mammalian and/or a tumor cell.

21. Cell line MC.Fas⁻ (date of deposition at DSMZ: 4 May 2006).

22. A method for preventing and/or treating fungal infections in a patient in need of such prevention and/or treatment, comprising administering to said patient an effective amount of an inhibitor of the Gliotoxin (GT)-mediated activation of the proapoptotic Bcl-2 family member Bak.
